(19) United States
(12) Patent Application Publication
(10) Pub. No.: US 2017/0311886 A1
(43) Pub. Date: Nov. 2, 2017

(54) A WEARABLE SENSING ASSEMBLY

(71) Applicant: Cambridge Temperature Concepts Ltd, Cambridge, Cambridgeshire (GB)

(72) Inventors: Oriane Elisabeth CHAUSIAUX, Cambridge (GB); Shamus Louis Godfrey HUSHEER, London (GB)

(21) Appl. No.: 15/520,656

(22) PCT Filed: Oct. 23, 2015
(86) PCT No.: PCT/GB2015/053185

§ 371 Date: Apr. 20, 2017

(30) Foreign Application Priority Data
Oct. 24, 2014 (GB) 1418985.6

(51) Int. Cl. A61B 5/00; A61B 5/01; A41C 3/00; A61B 5/021; A61B 10/00

(52) U.S. Cl. CPC A61B 5/6802; A61B 5/681; A61B 5/0008; A61B 5/01; A61B 10/0012; A41C 3/0064; A61B 5/6804; A61B 5/021; A61B 2010/0019; A61B 2560/0443; A61B 2562/029

(57) ABSTRACT

A sensing assembly having: a sensing device having first and second opposing major surfaces, a first sensor located in the device and positioned for preferentially sensing a parameter at the first major surface and a second sensor located in the device and positioned for preferentially sensing the parameter at the second major surface; and a wearable carrier for embracing a part of the human or animal body, the carrier having a hole extending fully therethrough; the sensing device and the carrier being cooperatively configured so that the sensing device can be releasably lodged in the hole in the carrier so that when the carrier is embracing the body part the sensing device can be held against the body part by the carrier with one major surface of the sensing device facing the body part and with no part of the carrier between the sensing device and the body.

This invention relates to wearable devices.

There is increasing interest in the field of wearable sensing devices for measuring a user's physiology, for sports or healthcare purposes. These devices typically include one or more sensors for monitoring parameters such as heart rate, blood pressure and temperature, a memory for storing the logged data and a communication interface for transmitting the logged data to another unit for analysis.

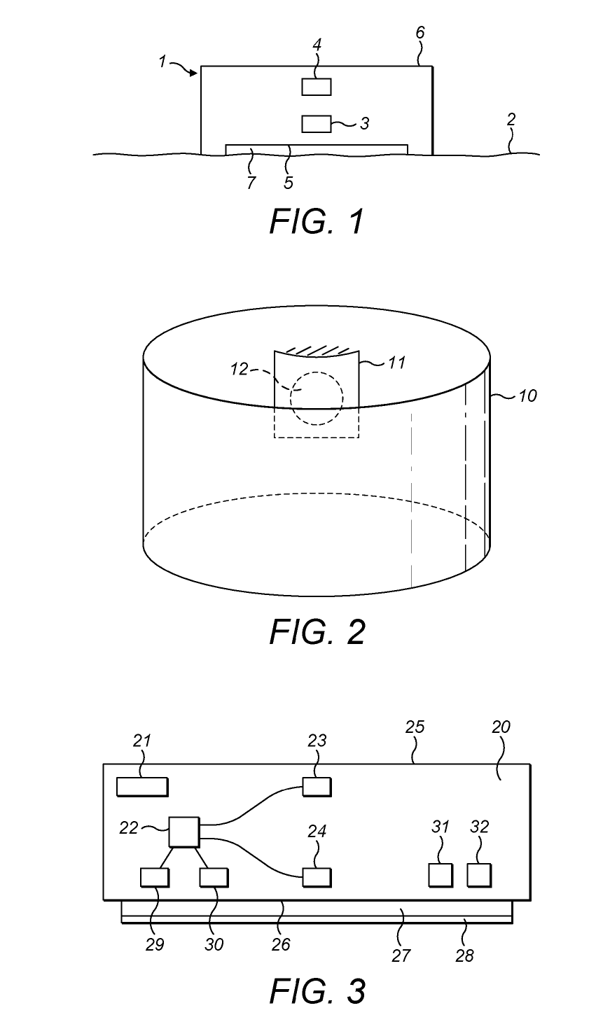

FIG. 1 shows an example of one such device. This device is for estimating the body temperature of a female user in order to make inferences about the timing of her point of ovulation. This information can be used to assist the user to conceive. In order to estimate the point of ovulation the user's body temperature must be sensed with considerable sensitivity. This calls for careful design of the sensing device. It has been found that a device of the type shown in FIG. 1 is particularly suitable. The device 1 of FIG. 1 is shown applied to the skin 2 of a user. The device has two temperature sensors 3, 4. Sensor 3 is located close to a first major surface 5 of the device, which faces the skin of the user. Sensor 4 is located close to a second major surface 6 of the device, which is exposed. By analysing the difference between the measurements from the two temperature sensors, estimates can be made of the changes in the user's body temperature that are sufficiently accurate to allow the user's point of ovulation to be predicted.

Temperature sensors of this type raise specific problems in how they are held in contact with a user, due to the need to take highly accurate temperature measurements.

To get the best results it is important for the first major surface 5 of the device to be firmly applied to the user's skin. If an air gap develops from time to time between the first major surface and the skin then that can impair the measurement accuracy. One way to keep the first major surface applied to the skin is by providing a layer of adhesive 7 on the first major surface, which sticks the major surface to the user's skin.

The adhesive can be chosen to be of a composition and thickness that provides good adhesion and good thermal conductivity. A problem with this, however, is that some users are sensitive to certain adhesives. For those users, an adhesively applied sensor can cause a rash to develop in the area of skin where the device has been adhered.

There is a need for an improved way of attaching such a sensor against a user's skin.

One option would be to use an adhesive that is formulated for reduced dermatological irritation. However, such adhesives are generally less effective in adhering the sensor to the skin, may compromise heat conduction and may still not avoid sensitivity in all users.

Another known option is to provide a wearable element that incorporates a pouch in which the device can be held. FIG. 2 shows an example of such an element, in the form of an elasticated armband. The armband is formed of an annular band 10 of elastic fabric which can be fitted around the upper arm of a user. The band incorporates a pouch 11 defined by an additional layer of fabric on the interior of the band. The pouch is open at one end so a device 12 can be inserted into the pouch. When the band is worn it holds the device near the user's skin. A problem with this approach is that the fabric of which the band and the pouch are formed prevents the inner sensor of the device from making good contact with the skin and prevents the outer sensor of the device from being properly exposed to the exterior. This has been found to reduce the quality of the results.

A vast range of alternative mechanisms have been developed for attaching physiological monitoring devices to the body. These include garments incorporating connections to sensing devices (see, for example, WO 2009/130595 and US 2013/0281795), various wrist strap designs (see, for example, US 2011/0007468), elastic chest straps including elements such as press studs for connection to sensing devices (see, for example, US 2013/0131484), devices incorporated in clothing buttons, embroidery or fabrics (see, for example, WO 2005/103923), devices that clip to conventional items of clothing such as bras (see, for example, U.S. Pat. No. 8,560,044) and articles that incorporate sensing devices and are configured as hooks that can be engaged with a user's ear (see, for example, US 2009/0227853). However, it is believed that the prior art does not address the problem of positioning temperature sensing devices for improved accuracy without the use of adhesive.

There is a need for a way of configuring a temperature sensing device so that it can perform with sufficiently high accuracy to provide a useful indication of a female's point of ovulation, and also so that it can be used by people who are sensitive to adhesive.

According to the present invention there is provided a sensing assembly having: a sensing device having first and second opposing major surfaces, a first sensor located in the device and positioned for preferentially sensing a parameter at the first major surface and a second sensor located in the device and positioned for preferentially sensing the parameter at the second major surface; and a wearable carrier for embracing a part of the human or animal body, the carrier having a hole extending fully therethrough; the sensing device and the carrier being cooperatively configured so that the sensing device can be releasably lodged in the hole in the carrier so that when the carrier is embracing the body part the sensing device can be held against the body part by the carrier with one major surface of the sensing device facing the body part and with no part of the carrier between the sensing device and the body.

The device and the carrier may be configured such that when the carrier is embracing the body part the sensing device can be held against the body part by the carrier with one major surface of the sensing device facing the body part and with no part of the carrier between the sensing device and the body and no part of the carrier between the sensing device and the region external to the carrier.

The sensing device may have an exterior wall encircling the device between the first and second major surfaces. The hole in the carrier may have an interior wall defining the interior surface of the hole. The exterior wall and the interior wall may be cooperatively configured to mate resiliently with each other.

The interior wall may encircle the hole or extend only partially around it.

The sensing device and the carrier may be cooperatively configured so that the sensing device can be retained in the hole in the carrier.

The sensing device may include a channel running at least partially around its exterior between the first and second major surfaces, and the interior wall of the carrier may be configured to be retained in the channel.

The wearable carrier may be a strap for engaging a wrist, leg or chest of a user.

The wearable carrier may be an undergarment.

The parameter may be temperature and the sensors may be temperature sensors.

The parameter may be humidity and the sensors may be humidity sensors.

The sensing device may comprise a processor configured for estimating the parameter at or beyond the surface of the device in dependence on both the parameter as sensed by the first sensor and as sensed by the second sensor.

The sensing device may comprise a transceiver and the transceiver may be configured for transmitting to a receive external to the device data representing one or more values sensed by the device.

The carrier may comprise electrodes exposed on its inner surface for contacting the skin of a wearer of the carrier and conductors extending from the electrodes to the surface of the hole in the carrier. The sensing device may comprise electrical contacts for contacting the conductors and an electrical sensor for sensing a physical parameter of a wearer of the carrier by means of the electrodes.

The carrier may comprise an optical window exposed on its inner surface for optical communication with the skin of a wearer of the carrier and an optical carrier extending from the window to the surface of the hole in the carrier.

The sensing device may comprise an optical window for communicating with the optical carrier and an optical sensor for sensing a physical parameter of a wearer of the carrier by means of the optical window of the carrier.

The first sensor may be located adjacent the first major surface and the second sensor may be located adjacent the second major surface.

The carrier may be elastically extendible for maintaining the sensing device in contact with the skin of a wearer when the sensing device is located in the hole in the carrier and the carrier is worn on the body part.

The carrier may be configured to embrace the body part.

The carrier may comprise a releasable clasp by means of which the carrier can adopt any of a range of circumferences for engagement around the body part.

The present invention will now be described by way of example with reference to the accompanying drawings. In the drawings:

FIG. 1 is a schematic cross-section through a temperature sensing device attached to a user's skin.

FIG. 2 shows an armband for carrying a temperature sensing device.

FIG. 3 is a schematic cross-section through a temperature sensing device.

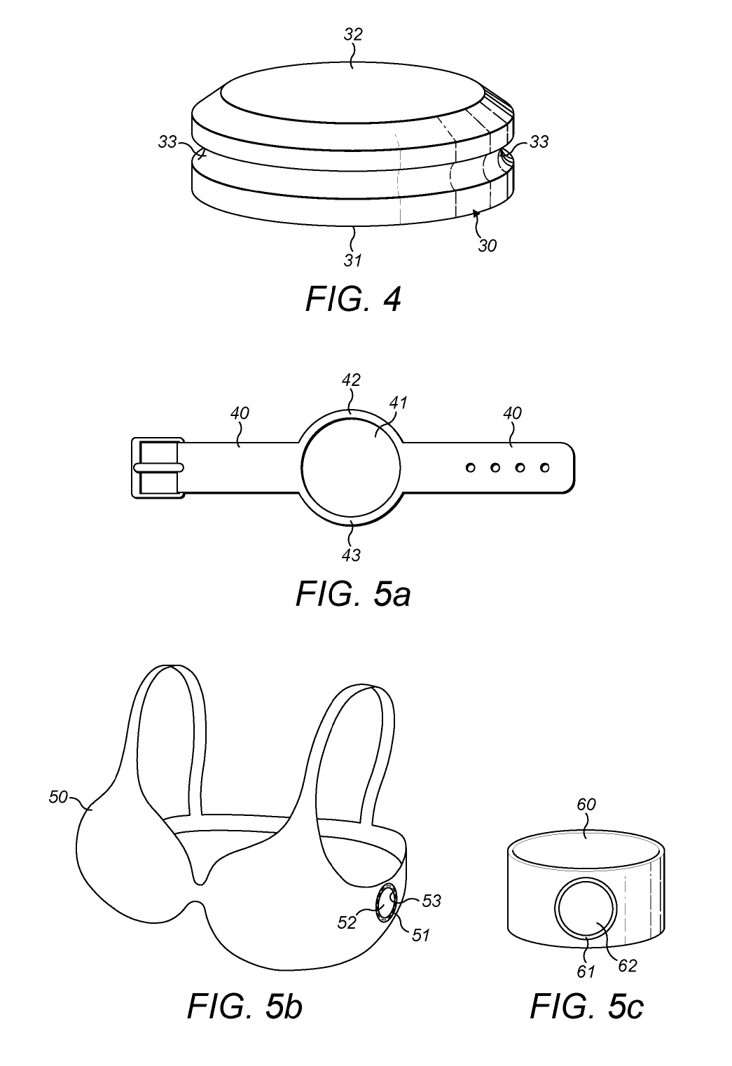

FIG. 4 is an isometric view of a temperature sensing device having a circumferential channel.

FIG. 5 shows accessories and garments that can physically couple to the device of FIG. 4.

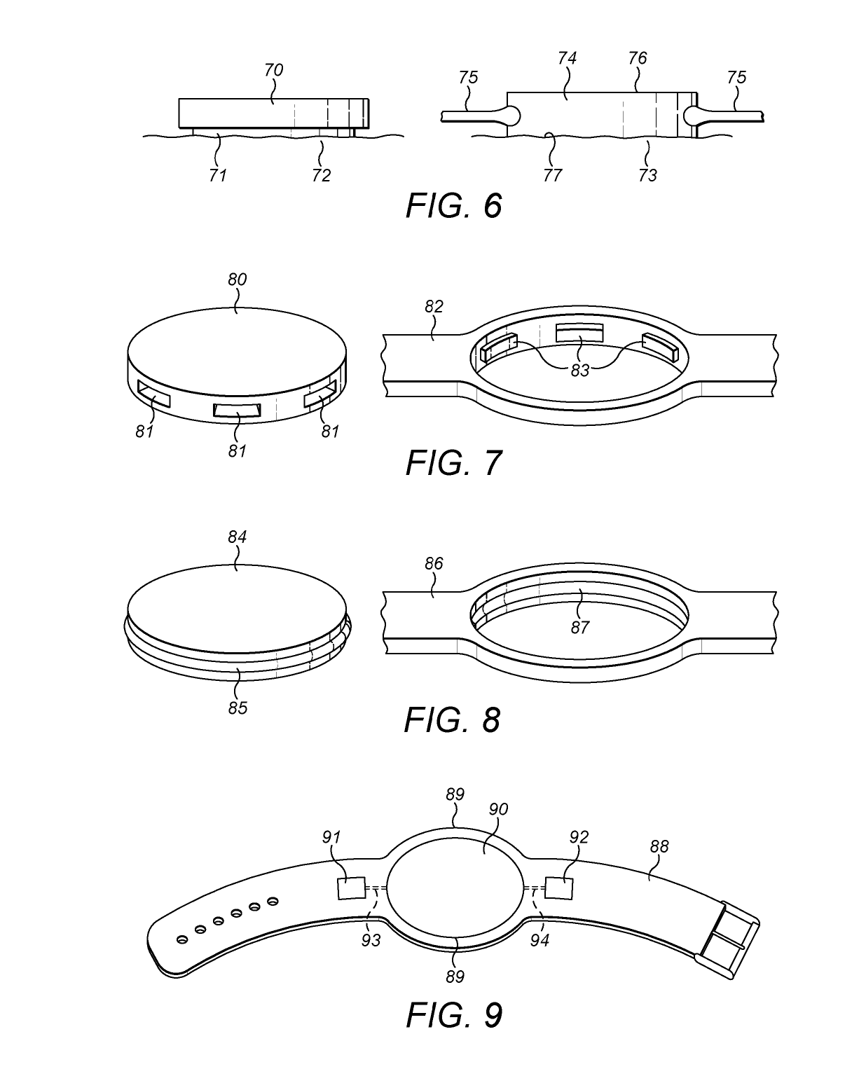

FIG. 6 shows cross-sections through temperature sensing devices.

FIG. 7 shows a temperature sensing device having a set of peripheral notches, and an associated wristband.

FIG. 8 shows a temperature sensing device having a circumferential ridge, and an associated wristband.

FIG. 9 shows a wristband having integrated electrodes.

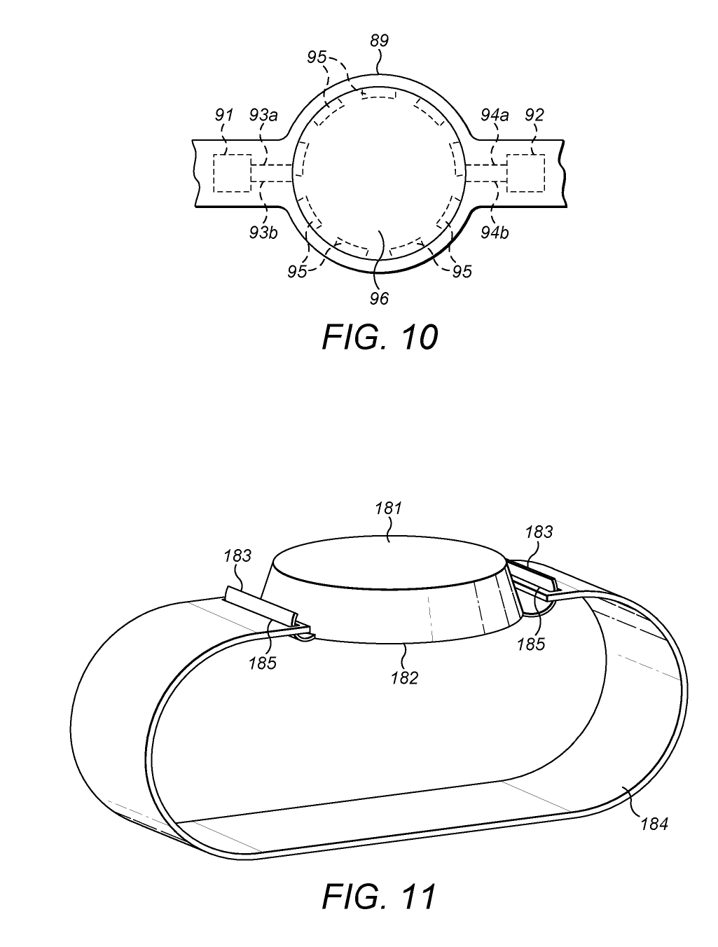

FIG. 10 shows an electrode arrangement for communication between the wristband of FIG. 9 and a sensor device.

FIG. 11 shows another arrangement of sensor device and carrier strap.

A wearable article such as a wriststrap or undergarment can be provided with a strap or panel in which there is a hole running from one side of the article to the other. The region surrounding the through-hole is configured to cooperate with a temperature sensing device so that the temperature sensing device can be releasably lodged in the through-hole. Since the hole extends through the article, the temperature sensing device, once lodged in the hole, can be held next to the skin of a person wearing the article. That means that heat can be transferred across the interface between the wearer's skin and the temperature sensing device without there being an intermediate element such as an adhesive pad or a sheet of the wearable article in the way. That can help the temperature sensing device to accurately measure the temperature of the wearer. Similarly, heat can be transferred between the temperature sensing device and the region exterior to the wearable article without there being an intervening element between the two. That can help the temperature sensing device to accurately measure the temperature of the exterior region.

FIG. 3 shows a schematic cross-section through a temperature sensing device. The device has a body 20. Within the body 20 are a battery 21, a central processor 22, temperature sensors 23, 24, a radio transceiver 29 and a non-volatile memory 30.

The battery powers the other components.

The processor is configured to execute program code stored in the non-volatile memory 30. The program code may be such as to cause the processor to collect sensed data from the temperature sensors 23, 24, store the data temporarily in another region of the memory 30 and then transmit it to a consumer of the data by means of the radio transceiver 29.

The temperature sensors 23, 24 may be thermistors or any other suitable temperature sensing device. One temperature sensor 23 is located adjacent to a first surface 25 of the device, which will be exposed when the device is attached to a user's skin. This allows sensor 23 to provide an indication of ambient temperature. The other temperature sensor 24 is located adjacent to a second surface 26 of the device, which is intended to be positioned next to the user's skin. This allows sensor 24 to provide an indication of the temperature of the user's body. Each temperature sensor provides an output that is indicative of the sensed temperature. That output passes to the processor 22, which processes it to form digital data indicative of the measured temperatures. That digital data can then be stored in memory 30. In one example, the sensors may provide analog temperature outputs, and the processor may process them by analog-to-digital conversion.

The processor may perform processing on the sensed temperatures so as to convert from the sensed temperature, or a series of sensed temperatures over time, to an estimated true temperature. This may be performed using any of the techniques known in the art, which may be as simple as applying a predetermined offset to the sensed temperature or as complex as a full analysis of the general heat equation.

The transceiver 29 could be a short range transceiver. It could, for example, operate according to a protocol such as Bluetooth, IEEE 802.11 or ANT. The transceiver can operate under the control of the processor 22 to transmit data that has been stored in the memory to another device for analysis. That other device could be a computer or smartphone. The transceiver could be suitable for supporting a wired interface such as USB, in which case the device could have a suitable physical connector incorporated in it.

The device may include sensors 31, 32 for sensing other parameters. Those other parameters could be characteristics of the ambient environment, such as ambient light, air pressure, humidity, concentration of an atom, molecule or ion (e.g. of chlorine) or sound, or characteristics of the user, for example blood pressure, pulse rate, blood oxygen content and so on. Some examples will be discussed in more detail below. The outputs of those sensors are passed to the processor 22 for storing in the memory 30 and/or uploading by means of the transceiver 29. In each case the device could include two sensors, one exposed to each major surface for establishing a comparison of the measured property as presented on the surface of the wearer's body with the same property in the local environment. For example, there could be a humidity sensor on each side of the sensor for comparing humidity on the wearer's skin with atmospheric humidity, or a chlorine sensor for sensing chlorine concentration on the wearer's skin with chlorine concentration in the atmosphere.

In this example the user-facing, inner surface 26 of the device is coated with an adhesive layer 27 which is covered with a protective sheet 28. When the device is to be adhered to a user the protective sheet is peeled off, exposing the adhesive. The device can then be stuck to the user's skin. However, when the device is issued to a user the adhesive layer and the protective sheet could be separate from the device. The user can then choose either: (i) to apply the adhesive layer and stick the device to their skin, or (ii) to leave the device without the adhesive layer and apply the device by one of the alternative mechanisms described below. When the device is applied by those alternative mechanisms it may be possible to achieve better measurement results and/or to reduce the chance of skin irritation due to the absence of the adhesive.

The body 20 could be hollow. More preferably it could be filled with an insulating material such as rubber or silicone to help protect the components inside it. For ease of manufacture the internal components could be attached to one or more circuit boards. In FIG. 3 the body 20 is shown schematically as a block of rectangular cross-section. In practice the body could be shaped to help it to be held against the body, as will be discussed in more detail below.

FIG. 4 shows one example of how the exterior of the device could be configured. In FIG. 4 the body of the device is indicated generally at 30. The user-facing surface is shown at 31 and the surface to be exposed is shown at 32. There is a groove or channel 33 running around the periphery of the device. The groove runs between the user-facing surface 31 and the external surface 32. In cross-section the groove may be of any shape, but it is convenient if it is of part-circular shape since that avoids any sharp convex corners that may be difficult to clean. In plan the groove may again be of any shape, but it is convenient if it is of circular cross-section. Then the device can be fitted into a holder in any orientation.

FIG. 5 shows examples of articles that can mate with the temperature sensing device of FIG. 1. FIG. 5a shows a wrist holder. The wrist holder comprises a strap 40. The strap can encircle a user's wrist and be buckled (or otherwise fixed) into a loop. Part-way along the strap is a configuration for mating to the sensor device. The configuration comprises two bridges 42, 43 which diverge from the main elements of the strap leaving an opening 41 between them. The opening extends all the way through the wrist holder. The bridges are sized and shaped so that they can clip snugly into the circumferential groove 33 of the sensor device. One or both of the bridges may be resilient, for example by being formed of elastic material, in order that it or they can be stretched over the radially outermost parts of the sensor device and then snap into place in the circumferential groove, holding the sensor in place.

Since the hole 41 extends fully through the wrist holder, once the sensor device is attached in place there is no part of the holder to block the user-facing surface 31 of the sensor from being pressed against the user's skin and no part of the holder to block the external surface 32 of the sensor from being exposed to ambient conditions. This characteristic makes this holder especially suitable for use with a temperature sensor of the type shown in FIG. 4. Using a holder of this type more accurate temperature measurements can be obtained than by using an adhesive sheet, as shown in FIG. 3, since there is nothing to obstruct heat flow from the body of a user to the device. This is illustrated in more detail in FIG. 6. In the left-hand portion of FIG. 6 a first sensing device 70 is shown adhered by an adhesive sheet 71 to the skin 72 of a user. The adhesive sheet separates the sensor from the skin. In the right-hand portion of FIG. 6 a sensing device 74 generally of the type shown in FIG. 4 is shown held against a user's skin by a strap 75. The strap 75 engages the sensing device 74 around its periphery, and does not cover the outer or inner surfaces 76, 77 of the sensing device. As a result, the sensing device of FIG. 6 can be held intimately against the skin of the user, improving thermal conductivity between the skin and the sensing device. Similarly, the outer surface of the sensing device is not occluded by a sheet, as in the arrangement of FIG. 2.

By having one side of the temperature sensing device directly exposed to the user's skin and the other directly exposed to the environment the temperature sensing device is able to take relatively accurate measurements of the temperature of the skin and the environment around the sensor. Having those two measurements allows the processor 22 to derive a more accurate estimate of the user's body temperature. The user's skin temperature tends to be elevated when the adjacent environment is warmer. When measurements of the skin temperature and the environmental temperature are available the measured skin temperature (T_{S}) can be adapted in dependence on the measured environmental temperature (T_{E}) to derive an estimate of the user's body temperature (T_{B}). In a simple example, T_{B} may be estimated as T_{S}+(a−T_{E})/b where a and b are experimentally derived constants. In practice, the adaptation could be performed by using experimentally derived lookup table.

FIG. 5b shows a bra 50 having an attachment 51 for the sensor of FIG. 4. The attachment defines a resilient annulus having a through-hole 52 at its centre. The resilient annulus is stitched or otherwise adhered to the fabric of the bra. The inward-facing wall 53 of the annulus is sized to sit within the peripheral groove 33 of the sensor. Due to its resilience the inward facing wall 53 can be drawn over the outermost parts of the sensor 30 and the wall can then be snapped in place in the peripheral groove 33, holding the sensor device in place in the annulus. When the sensing device is clipped in this way into an undergarment such as a bra the inward facing surface of the sensing device can be exposed to and in firm contact with the user's skin. That permits good thermal conductivity between the skin and the sensing device.

FIG. 5c shows a wrist-band 60 having a circular hole 62 therethrough. The rim 61 of the circular hole is resilient and shaped to snap fit into the circumferential groove 33 of the temperature sensing device 30. The sensing device can be snapped into engagement with the rim 61, allowing it to be held against the skin of a user when the wristband is being worn.

The watch strap has a buckle that can be engaged in any of a number of holes in the strap so it can adopt any of a plurality of circumferences and thereby fit snugly around the wrist. In addition the strap may be fully or partially elastic to maintain tension around the wrist and thereby keep the temperature sensor against the skin of a wearer. The bra has a hook and eye arrangement for adjustment around the torso of a wearer to achieve a similar result. Either carrier may be equipped with clasps of different types.

In the examples given above, the holder for the temperature sensing device is a configuration that can be snap fitted into the groove 33. Other arrangements are possible. In one alternative arrangement, instead of having a peripheral channel that extends fully around the temperature sensing device, the device could have a series of indentations around its periphery between its major surfaces. Those indentations could mate with corresponding male configurations on the interior wall of the hole. This is illustrated in FIG. 7, which shows a temperature sensing device 80 having peripheral indentations 81, and a strap 82 having a hole therethrough and protrusions 83 on the interior wall of the hole which can mate with the indentations on the sensing device. In another alternative arrangement, instead of the sensing device being provided with one or more female configurations and the inner wall of the hole being provided with one or more male configurations that can mate with the female configurations of the sensing device, the sensing device could be provided with one or more male configurations and the inner wall of the hole could be provided with one or more female configurations that can mate with the male configurations of the sensing device. This is illustrated in FIG. 8, which shows a temperature sensing device having a circumferential ridge 85, and a strap 86 having a hole therethrough and a channel 87 running around the interior wall of the hole which can mate with the circumferential ridge on the sensing device. In other examples, the sensor could mate with the material surrounding the through-hole (e.g. 41, 52, 62) by a mechanism other than snap fitting. For example, the sending device could be a screw fit or bayonet fit in the material surrounding the through-hole. The carrier for the sensing device could be configured so that there is a magnetic interaction between the sensing device and the carrier which causes the sensing device to be held in place in the carrier. Instead of or in addition to having a groove around its periphery the sensing device could have a formation that projects radially outwards and that snap fits into a corresponding groove in the carrier. The sensing device could engage with the carrier by virtue of removable pins or other elements that can pass through the carrier and into the sensing device to lock the two together. In a further example, the interior wall of the hole could be bounded by radially inwardly extending flanges, one of which may be resilient, between which the sensing device can be clipped.

It is preferred that the sensing device is circularly symmetrical about an axis perpendicular to the face that is to be positioned against a user's skin. That allows the device to be positioned in the carrier in any orientation about that axis. However, the sensing device could be of any other suitable shape.

In one embodiment the interface between the sensing device and the carrier may be circular and planar, so that the sensing device can be attached to the carrier in any orientation about an axis perpendicular to its major plane. In another embodiment, the sensing device may be configured to accept the carrier about an interface that is noncircular parallel to the major plane of the device, or that deviates from a single plane. In that way the interface can maintain a fixed orientation between the sensing device and the carrier. This is useful in applications where the sensor makes measurements that are directional in the plane of the sensor faces, for example in the case of a three-axis accelerometer worn on the wrist, wherein by suitable orientation of the sensor one axis of the accelerometer could point out of the body whilst another axis could point along the length of the arm.

It is preferred that the sensing device is relatively thin: for example having a distance of less than 10 mm, 8 mm, 6 mm or 4 mm between its inward and outward temperature sensing faces. That makes it more convenient for wearing. It is preferred that the inward and/or the outward temperature sensing faces are generally parallel to each other.

In the examples of FIGS. 5a to 5c the hole for holding the temperature sensing device is located in an element of a wearable article. In each of those examples, the element in which the hole is located has sufficient width parallel to its interface with the body that it will resist twisting of the element about an axis parallel with the skin. That can be advantageous because it can permit the element to also resist rotation of the temperature sensing device about such an axis. Rotation of that type could reduce the degree of contact between the temperature sensing device and the wearer's skin, impairing the accuracy of the device's temperature measurements.

It is preferred that the major face of the temperature sensing device that faces the user's skin is flat or substantially flat. That makes for better contact with the user's skin. Conveniently the opposite major face may be flat or substantially flat. Alternatively, it could be convex or concave.

In the examples above the temperature sensing device is engaged with a wearable carrier such as a wristband or an undergarment such as a bra. The carrier could take other forms. It could be a strap for encircling another part of the body, for example a chest strap. It could be another form or undergarment, for example a sock, a vest, a shirt or a pair of underpants. It is preferably a garment that is sufficiently elastic or adjustable that it can be held snugly against the body of a wearer in order to keep the temperature sensing device applied to the skin of a user when the user moves around. The wearer of the carrier could be a human or an animal, conveniently a mammal.

It may be desirable for the combination of the sensing device and its carrier to be able to sense parameters other than temperature. For example, it may be desirable to sense the user's pulse or galvanic skin response. As is well known those parameters can be sensed by means of electrodes that are in contact with the user's skin. Those electrodes could be integrated into the major face of the sensing device that is to face the user's skin. Alternatively, they could be integrated into the carrier, the carrier could be configured so that there is a conductive path through the carrier from the electrodes to the inner rim of the hole where the sensing device can be mounted, and the sensor could be configured so that there are contacts on its periphery that can make contact with the conductive paths where they meet the inner rim of the hole. FIG. 9 shows a wrist strap 88 having conductive electrodes for contacting a user's skin when the strap is being worn. The strap is in two major parts which are joined by webs 89 so as to define a hole 90 through the strap. There are two zones 91, 92 of electrically conductive material exposed on the interior surface of the strap. Those zones can serve as electrodes for interfacing with a user's skin. Conductive leads 93, 94 extend through the material of the strap from the electrodes to the rim of the hole 90. FIG. 10 shows another example of such a strap with a temperature sensing device located in the hole. This example illustrates a mechanism for permitting the sensing device to make electrical contact with the electrodes on the strap irrespective of the orientation of the sensing device in the strap. In the example of FIG. 10 the electrodes are 91 and 92. Two leads 93a, 93b, 94a, 94b run from each electrode to the rim of the hole. The leads of each pair terminate at locations on the rim that are spaced apart from each other. The temperature sensing device 96 is provided with multiple electrical contacts 95 spaced around its outer surface. The contacts 95 are electrically insulated from each other by the body of the sensing device and are sized so that in any orientation of the sensing device when it is properly engaged in the hole in the strap at least one electrode 95 will be in contact with at least one of the leads 93a, 93b to electrode 91 and at least one electrode 95 will be in contact with at least one of the leads 94a, 94b to electrode 92. Once the sensing device is in operation it can try combinations of the contacts in succession until it determines which ones are in contact with the electrodes 91, 92. A simpler contact arrangement could be adopted if the sensor is only capable of being clipped into the carrier in a certain orientation.

A similar arrangement can be used for performing optical sensing on the wearer. In this arrangement the strap includes an optical window that opens on the interior surface of the strap. An optical fibre embedded within the strap permits optical communication between that window and a second window that opens on the interior rim of the hole where the sensing device can be mounted. On the sensing device there is an optical emitter and sensor that can be positioned adjacent the second window. The optical emitter and sensor can be of the type conventionally used for optical sensing of physical parameters of a human or animal, for example for pulse oximetry. This arrangement allows the wearer's body to be sensed at a location remote from the location of the sensing device itself. This can be useful, for example, when the sensing device is mounted on a wrist strap. Many people find it most comfortable to wear a wrist-mounted device on the anterior surface of the wrist, whereas blood vessels are generally more accessible for optical sensing on the posterior surface of the wrist. Using the strap described above, the sensing device can be positioned on the anterior surface whilst the blood vessels are sensed on the posterior surface.

The strap could be in the form of an elastically extendible loop, so that it can be stretched over the hand of a wearer and contract to lie on the wearer's wrist. Alternatively the strap could be separable and one or both ends of the strap could be equipped with fixings such as a buckle, a clip, an eye for receiving a tang or stud borne by the other end of the strap, hook and/or loop fastenings or a magnetic clasp.

The straps and other carriers shown in the figures include a single opening (e.g. 41, 62, 90). The carrier could include multiple such openings for housing multiple sensor devices.

The carrier embraces a part of the user's body so as to retain the sensor device in place there on. It could do so by encircling the body part or passing partially around the body part so as to clip on to the body part.

The carrier may fully or partially encircle the zone where the sensor device is to be retained. The sensor device and the carrier may be configured so that the sensor device can be releasably retained in the carrier. The device may clip resiliently into the zone in the carrier, e.g. by snap fitting.

In another embodiment, instead of a strap having a hole therethrough in which the temperature sensing device can be attached, the temperature sensing device could have attachment points on opposite sides, to each of which a respective length of a strap can be attached. Then the strap could be looped around a body part and the two lengths of the strap joined together. The attachment points could, for example, be hooks that could attach to eyes on the strap lengths, or eyes that could attach to hooks on the strap lengths. Since this embodiment also permits minimally obstructed or unobstructed contact between the sensor device and both the wearer's skin and the environment, it too permits accurate measurements of a parameter on the skin and in the environment, for example to allow a measurement of the parameter on the user's skin to be calibrated against a corresponding measure with respect to the environment. FIG. 11 shows an example of such an arrangement. The arrangement comprises a sensor device 180, which is arranged with internal sensors for sensing a common parameter (e.g. temperature or humidity) at an exterior surface 181 and an interior surface 182. The device comprises a pair of cupped attachment points 183 extending from its periphery. A strap 184 for embracing the body of a user is provided with loops 185 at its ends. The loops pass over the attachment points, enabling the strap to hold the sensor device against the wearer's body. The strap could be provided with a buckle or clasp. The strap could be elastically extensible. The strap could be sized to fit around a desired part of the wearer's anatomy: for example the chest, leg or arm, including the wrist.

The sensor device may comprise other sensors. For example, it may comprise an infra-red sensor for sensing incident light on the exterior-facing major surface, electrodes in the interior-facing major surface coupled to a sensor capable of detecting galvanic skin response, or an accelerometer capable of detecting acceleration of the sensor device. Such an accelerator may be a tri-axis accelerometer: that is one that is capable of detecting accelerations along three non-parallel axes. The tri-axis accelerometer may detect accelerations along three mutually orthogonal axes. Preferably one of those axes is perpendicular to the plane of the skin-facing major surface.

By enabling the sensor to be readily attached to and detached from the carrier, the sensor can be used either on the carrier or detached from the carrier and adhered by adhesive to the user's skin.

Some non-limiting examples of uses to which data derived from the sensor may be put are assisting natural conception, natural contraception, artificial insemination, in-vitro fertilisation (IVF), detecting or predicting ovulation, skin care, assisting post-operative recovery and diagnosis, assisting weight management, baby monitoring, monitoring sports performance, monitoring performance in extreme environments, tamper evidence, wearer tracking, in-hospital monitoring of bodily functions, assisting fitness, health, wellbeing or activity management, and detection, diagnosis, treatment, management or background monitoring for of any of the following conditions: chronic obstructive pulmonary disease (COPD), cystic fibrosis (CF), diabetes, hypoglycaemia, sleep disturbance, sleep apnoea, chronic pain, infection (e.g. by bacterial, viral, prion, protozoal, fungal or parasitic agents), sepsis, polycystic ovary syndrome (PCOS), menopause, asthma, insomnia, schizophrenia, coronary heart disease, narcolepsy, restless legs syndrome, rheumatoid arthritis, inflammatory bowel disease (IBD), lupus, periodic fever syndromes and cancers such as lymphoma, leukaemia and renal cancer. The sensor and the carrier may be applied to humans or animals.

The sensor device may be used to estimate the flux of a quantity such as temperature or humidity between the wearer and the environment. Since the sensors can be configured to sensitively measure the relevant quantity on the wearer's skin and in the adjacent environment, an estimate of the relevant flux can more readily be obtained than with sensor arrangements in which either of those measurements is obscured.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A sensing assembly having:
a sensing device having first and second opposing major surfaces, a first sensor located in the device and positioned for preferentially sensing a parameter at the first major surface and a second sensor located in the device and positioned for preferentially sensing the parameter at the second major surface; and
a wearable carrier for embracing a part of the human or animal body, the carrier having a hole extending fully therethrough;
the sensing device and the carrier being cooperatively configured so that the sensing device can be releasably lodged in the hole in the carrier so that when the carrier is embracing the body part the sensing device can be held against the body part by the carrier with one major surface of the sensing device facing the body part and with no part of the carrier between the sensing device and the body.

2. A sensing assembly as claimed in claim 1, wherein the device and the carrier are configured such that when the carrier is embracing the body part the sensing device can be held against the body part by the carrier with one major surface of the sensing device facing the body part and with no part of the carrier between the sensing device and the body and no part of the carrier between the sensing device and the region external to the carrier.

3. An assembly as claimed in claim 1, wherein:
the sensing device has an exterior wall encircling the device between the first and second major surfaces;
the hole in the carrier has an interior wall defining the interior surface of the hole; and
the exterior wall and the interior wall are cooperatively configured to mate resiliently with each other.

4. An assembly as claimed in claim 3, wherein the interior wall encircles the hole.

5. An assembly as claimed in claim 3, wherein the interior wall extends partially around the hole.

6. An assembly as claimed in claim 1, wherein the sensing device and the carrier are cooperatively configured so that the sensing device can be retained in the hole in the carrier.

7. An assembly as claimed in claim 6, wherein the sensing device includes a channel running at least partially around its exterior between the first and second major surfaces, and the interior wall of the carrier is configured to be retained in the channel.

8. An assembly as claimed in claim 1, wherein the wearable carrier is a strap for engaging a wrist, leg or chest of a user.

9. An assembly as claimed in claim 1, wherein the wearable carrier is an undergarment.

10. An assembly as claimed in claim 1, wherein the parameter is temperature and the sensors are temperature sensors.

11. An assembly as claimed in claim 1, wherein the parameter is humidity and the sensors are humidity sensors.

12. An assembly as claimed in claim 1, wherein the sensing device comprises a processor configured for estimating the parameter at or beyond the surface of the device in dependence on both the parameter as sensed by the first sensor and as sensed by the second sensor.

13. An assembly as claimed in claim 1, wherein the sensing device comprises a transceiver and the transceiver is configured for transmitting to a receive external to the device data representing one or more values sensed by the device.

14. An assembly as claimed in claim 1, wherein:
the carrier comprises electrodes exposed on its inner surface for contacting the skin of a wearer of the carrier and conductors extending from the electrodes to the surface of the hole in the carrier; and
the sensing device comprises electrical contacts for contacting the conductors and an electrical sensor for sensing a physical parameter of a wearer of the carrier by means of the electrodes.

15. An assembly as claimed in claim 1, wherein:
the carrier comprises an optical window exposed on its inner surface for optical communication with the skin of a wearer of the carrier and an optical carrier extending from the window to the surface of the hole in the carrier; and
the sensing device comprises an optical window for communicating with the optical carrier and an optical sensor for sensing a physical parameter of a wearer of the carrier by means of the optical window of the carrier.

16. An assembly as claimed in claim 1, wherein the first sensor is located adjacent the first major surface and the second sensor is located adjacent the second major surface.

17. An assembly as claimed in claim 1, wherein the carrier is elastically extendible for maintaining the sensing device in contact with the skin of a wearer when the sensing device is located in the hole in the carrier and the carrier is worn on the body part.

18. An assembly as claimed in claim 1, wherein the carrier is configured to embrace the body part.

19. An assembly as claimed in claim 18, wherein the carrier comprises a releasable clasp by means of which the carrier can adopt any of a range of circumferences for engagement around the body part.

20. (canceled)
